(19) United States
(12) Patent Application Publication
(10) Pub. No.: US 2009/0005367 A1
(43) Pub. Date: Jan. 1, 2009

(54) COMPOSITION AND METHOD FOR TREATING ALCOHOLISM AND OTHER SUBSTANCE ADDICTIONS

(76) Inventor: Murray James Propes, Chicago, IL (US)

Correspondence Address: MICHAEL BEST & FRIEDRICH LLP, ONE SOUTH PINCKNEY STREET, P O BOX 1806, MADISON, WI, 53701, US

(21) Appl. No.: 11/851,634

(22) Filed: Sep. 7, 2007

Related U.S. Application Data
(60) Provisional application No. 60/910,017, filed on Apr. 4, 2007.
(60) Provisional application No. 60/828,830, filed on Oct. 10, 2006.

(51) Int. Cl. A61K 31/5513; A61K 31/5517

(52) U.S. Cl. 514/220; 514/221

(57) ABSTRACT

Disclosed are pharmaceutical compositions comprising a benzodiazepine, an alcohol aversive agent, and an abuse aversive agent, and methods of use in preventing alcohol or cocaine abuse.

### CROSS REFERENCE TO RELATED APPLICATIONS

This application claims priority to U.S. Provisional Application No. 60/910,017, filed Apr. 4, 2007, and to U.S. Provisional Application No. 60/828,830, filed Oct. 10, 2006, each of which is incorporated by reference in its entirety.

### STATEMENT REGARDING FEDERALLY SPONSORED RESEARCH AND DEVELOPMENT

Not applicable.

### INTRODUCTION

The National Institute on Alcohol Abuse and Alcoholism (NIAAA) most recently estimates that 19.4 million or 9.4% of adult Americans are affected by alcohol or other substance use disorders¹. Alcoholism is estimated to cost our economy at least 148 billion dollars annually. Additionally, an estimated 18,000 people are killed in the US each year in automobile accidents involving motorists driving under the influence (DUI), which accounts for 39% of automobile-related fatalities, and 1,400,000 citations for DUI are issued annually. Alcoholism adversely impacts families of alcoholics in myriad ways. For example, alcoholism is associated with increased incidence of domestic violence and poverty. Offspring of women who consume alcohol during pregnancy are at risk for developing fetal alcohol syndrome.

Even when faced with loss of health, family, and livelihood from continued drinking, most alcoholics are simply unable to stop drinking, i.e. they are addicted to alcohol. Many patients are required to maintain absolute sobriety in order to be eligible to receive treatment for other health issues, for example, treatment in preparation for a liver transplant, or treatment for viral hepatitis. Failure to maintain sobriety leads to denial of treatment and premature death. In addition to the psychological components of addiction, and the initial acute physical withdrawal symptoms (tremors, hallucinations, nausea), many patients suffer persistent symptoms of anxiety and insomnia, which makes continued sobriety very difficult to maintain.

Disulfiram, or bis(diethylthiocarbamoyl)disulfide (Antabuse®) is an agent that causes alcohol intolerance and was approved by the FDA in 1951 for the treatment of alcoholism. However, its use and effectiveness have been limited because of poor compliance²⁻³. Disulfiram acts by blocking aldehyde dehydrogenase, thereby inhibiting oxidation of alcohol to acetic acid and resulting in the accumulation of acetaldehyde to concentrations 5 to 10 times higher than that found following ingestion of the same amount of alcohol in the absence of disulfiram. The accumulation of acetaldehyde produces a complex of unpleasant symptoms that prevents further alcohol intake, including flushing, sweating, headache, nausea, vomiting, tachycardia, palpitations, hyperventilation, hypotension, and dyspnea. Even minute amounts of alcohol are sufficient to produce an acute, unpleasant physical response, which can last from 30 minutes to several hours, and can occur up to two weeks following cessation of treatment with disulfiram.

Recently, disulfiram has been used in combination with naltrexone or acamprosate, two newer pharmacological agents approved for treatment of alcoholism. Naltrexone is an opioid-receptor antagonist approved for use in the treatment of alcohol dependence in conjunction with psychosocial interventions. Naltrexone is believed to work by blocking μ-opioid receptors, thereby reducing the reinforcing effects of alcohol, leading to reduced feelings of intoxication and fewer cravings. Acamprosate (calcium homotaurinate) is a structural analogue of y-aminobutyric acid (GABA), and is thought to decrease alcohol intake by affecting calcium channels and modifying transmission along GABA and glutamine pathways in the brain, which may result in decreased positive reinforcement of alcohol intake and decreased withdrawal cravings. However, studies examining the efficacy of disulfiram used in combination with naltrexone or acamprosate in the treatment of alcoholism demonstrate mixed results⁴⁻⁶.

There is an ongoing need for improved therapeutics and methods for treating alcoholism.

### BRIEF SUMMARY OF THE INVENTION

In one aspect, the present invention provides a pharmaceutical composition comprising a benzodiazepine, an alcohol aversive agent, and an abuse aversive agent.

In another aspect, the present invention provides a method of preventing alcohol consumption and reducing at least one symptom of alcohol withdrawal in a subject comprising administering to the subject the composition of the invention in an amount effective to reduce at least one symptom of alcohol withdrawal and to cause alcohol aversion.

The present invention also provides a method of preventing cocaine consumption in a subject comprising administering to the subject a pharmaceutical composition comprising a benzodiazepine and an alcohol aversive agent in an amount effective to reduce cocaine use.

### DETAILED DESCRIPTION OF THE INVENTION

The present invention provides a pharmaceutical composition comprising an alcohol aversive agent (e.g., disulfiram) and a benzodiazepine for use in the treatment of alcoholism, cocaine addiction, or other drug addiction. Benzodiazepines, like alcohol, have the pharmacological effect of increasing gabanergic transmission in the central nervous system, and are a preferred pharmacological agent for treatment of alcohol withdrawal⁷⁻⁸. The reinforcing properties of a benzodiazepine would be advantageous when combined with an alcohol aversive agent such as disulfiram for treatment of alcohol abuse. In other words, inclusion of a benzodiazepine in a pharmaceutical composition comprising an alcohol aversive agent is expected to promote compliance with treatment because it reduces the unpleasant symptoms associated with alcohol withdrawal. The pharmaceutical formulation further comprises a low (subclinical) dose of an abuse aversive agent such as atropine, an anticholinergic agent that causes dry mouth, tachycardia, and mydriasis, when taken in higher doses. Thus, including an abuse aversive agent such as atropine is expected to prevent abuse of the medicine.

Alcohol dependence has been observed to occur at a relatively high rate among people who abuse other addictive substances, such as marijuana, cocaine, and psychotherapeutics³⁴. Psychotherapeutics can include pain relievers, tranquilizers, stimulants and sedatives³⁴. Co-morbid alcohol-cocaine dependence has been associated with more severe dependence, poorer retention in treatment and poorer outcome with respect to either disorder alone. Some cocaine users report that the use of alcohol during a cocaine binge prolongs the cocaine “high”, relieves paranoia and stimulation during the binge, and diminishes acute abstinence symptoms.

Recent studies indicated that treatment of co-morbid alcohol and cocaine dependence with disulfiram afforded some benefit³²⁻³³. The reduced alcohol use was hypothesized to concomitantly reduce patient's exposure to alcohol, which can be a potent cue for cocaine use; lessening alcohol-related impairments in judgment and in the ability to resist craving and offers of drugs; and reducing exposure to cocaethylene, a pharmacologically active metabolite of cocaine and alcohol when they are used concurrently³¹. Results of further studies in which disulfiram was used to treat non-alcoholic patients addicted to cocaine indicate that disulfiram may be effective in treating cocaine addiction even in patients who are not alcohol dependent³¹. Therefore, in addition to treating alcoholism, the compositions and methods of the invention may be used to treat other drug addictions, including, but not limited to, cocaine addiction. Withdrawal from drugs such as cocaine can be associated with unpleasant side effects that may be reduced by administration of the compositions of the invention.

An “alcohol aversive agent” is an agent that causes unpleasant symptoms in people who are also consuming alcohol. As used herein, “alcohol” refers to ethanol. Disulfiram is a preferred alcohol aversive agent. Examples of other suitable alcohol aversive agents include, but are not limited to, citrated calcium carbamide (brand name Temposil) and coprine, a chemical found in certain mushrooms.

An “abuse aversive agent”, as used herein, is one that when taken at subclinical doses (i.e., at the prescribed amount), produces substantially no noticeable effects and which at higher doses (i.e., in amounts exceeding the prescribed dose), causes unpleasant or aversive effects (e.g., anticholinergic side effects, or nausea and vomiting). Atropine is a suitable abuse aversive agent. Other suitable abuse aversive agents include, but are not limited to, homatropine, guaifensin, and terpin hydrate.

A “subclinical dose” of an abuse aversive agent is a relatively low dose that does not typically produce unpleasant or aversive effects associated with higher doses of the abuse aversive agent.

An example of a pharmaceutical composition according to the present invention is a fixed-dose combination of clonazepam (a benzodiazepine), disulfiram (an alcohol aversive agent), and a subclinical dose of atropine, included as an abuse aversive agent. In the examples below is described a protocol that will be implemented to evaluate the efficacy of the pharmaceutical composition according to the present invention using a fixed dose combination comprising clonazepam (1 mg), disulfiram (250 mg), and atropine (0.025 mg) for oral administration.

Although a specific fixed dose combination will be used to evaluate the efficacy of administering an alcohol aversive agent together with a benzodiazepine and an abuse aversive agent in a single pharmaceutical composition in the treatment of alcoholism, one of skill in the art will appreciate that suitable pharmaceutical compositions according to the invention may include any suitable dose for each of the particular components comprised within the pharmaceutical composition. It is well within the ability of one skilled in the art to select appropriate dosages in developing pharmaceutical compositions according to the invention. The lower limit for the alcohol aversive agent is that which will cause an unpleasant effect in an individual ingesting alcohol. For the benzodiazepine, the lower limit is that which reduces at least one symptom of alcohol withdrawal.

In the examples below, the pharmaceutical composition is administered once daily. However, it is specifically envisioned that in the methods of the invention, the pharmaceutical composition may be administered at more than one time, i.e., the composition may be administered, for example, in two doses at different times of the day.

The composition is conveniently administered orally (e.g., as a tablet, capsule, liquid). However, any other suitable mode of administration is contemplated and encompassed within the scope of the present invention.

Preferably, the alcohol aversive agent, the benzodiazepine, and abuse aversive agent is provided in a formulation in which each of the components is substantially inseparable. In other words, a patient could not readily separate the formulation into its individual components.

Providing this composition to persons in need of eliminating alcohol consumption is expected to greatly improve patient compliance for treatment of alcohol use disorders and improve the patients' rehabilitation of chronic alcoholism. The pharmaceutical composition of the present invention may also comprise other agents, such as lactose, microcrystalline cellulose, corn starch, colloidal silicon dioxide, anhydrous lactose, magnesium stearate, microcrystalline cellulose, sodium starch glycolate, stearic acid, and the like.

The pharmaceutical combination is expected to be effective in treating patients with alcoholism. Preferably, the patient is a highly motivated problem drinker, binge drinker or has a history of any other alcohol use disorder. For those patients drinking more than 6 alcoholic drinks daily, the patient is preferably detoxified from alcohol prior to initiating treatment. Suitable patients include those who wish to stop drinking for medical or personal reasons, but who have been unable to do so. They may be trying to quit drinking for the first time, or may have tried other approved medical interventions (e.g., naltrexone, acamprosate, cognitive behavioral therapy, etc.), but due to the low clinical effectiveness of the currently approved therapies²⁹ and the widespread availability of alcohol, have relapsed.

Any suitable benzodiazepine may be used. Clonazepam is a preferred benzodiazepine because of its relatively low potential for abuse due to its pharmacokinetics (i.e. long elimination half-life of 30-40 hours). Non-limiting examples of other benzodiazepines that may be suitable for use in the present invention include: Alprazolam, Bromazepam, Chlordiazepoxide, Cinolazepam, Clobazam, Clonazepam, Clorazepate, Diazepam, Estazolam, Flunitrazepam, Flurazepam, Halazepam, Ketazolam, Loprazolam, Lorazepam, Lornetazepam, Medazepam, Midazolam, Nitrazepam, Nordazepam, Oxazepam, Prazepam, Quazepam, Temazepam, Tetrazepam and Triazolam (See Table 1).

TABLE 1

| | | Elimination Half- | | Approximate | |
|---|---|---|---|---|---|
| | Common Brand | Life (h)** [active | Primary | Equivalent | |
| Drug Name | Names* | metabolite] | Effects | Dose*** | |
| Alprazolam | Xanax, Xanor, Tafil, | 6-12 hours | anxiolytic | 0.5 | mg |
| | Alprox | | | | |
| Bromazepam | Lexotan, Lexomil, | 10-20 hours | anxiolytic | 5-6 | mg |
| | Somalium, Bromam | | | | |
| Chlordiazepoxide | Librium, Tropium, | 5-30 hours [36-200 | anxiolytic | 25 | mg |
| | Risolid, Klopoxid | hours] | | | |
| Cinolazepam | Gerodorm | 9 h | sedative | | |
| Clobazam | Frisium | 12-60 hours | anxiolytic, | 20 | mg |
| | | | anticonvulsant | | |
| Clonazepam | Klonopin, Klonapin, | 18-50 hours | anxiolytic, | 0.5 | mg |
| | Rivotril | | anticonvulsant | | |
| Clorazepate | Tranxene | [36-100 hours] | anxiolytic, | 15 | mg |
| | | | anticonvulsant | | |
| Diazepam | Valium, Apzepam, | 20-100 hours | anxiolytic | 10 | mg |
| | Stesolid, Apozepam, | [36-200] | | | |
| | Hexalid, Valaxona | | | | |
| Estazolam | ProSom | 10-24 h | hypnotic | 1-2 | mg |
| Flunitrazepam | Rohypnol, Fluscand, | 18-26 hours | hypnotic | 1 | mg |
| | Flunipam, Ronal | [36-200 hours] | | | |
| Flurazepam | Dalmane | [40-250 hours] | hypnotic | 15-30 | mg |
| Halazepam | Paxipam | [30-100 hours] | anxiolytic | 20 | mg |
| Ketazolam | Anxon | 2 hours | anxiolytic | 15-30 | mg |
| Loprazolam | Dormonoct | 6-12 hours | hypnotic | 1-2 | mg |
| Lorazepam | Ativan, Temesta, | 10-20 hours | anxiolytic | 1 | mg |
| | Lorabenz | | | | |
| Lormetazepam | Noctamid, Pronoctan | 10-12 hours | hypnotic | 1-2 | mg |
| Medazepam | Nobrium | 36-200 hours | anxiolytic | 10 | mg |
| Midazolam | Versed, Hypnovel | 3 hours (1.8-6 | auxiolytic | 5-15 | mg |
| | | hours) | | | |
| Nitrazepam | Mogadon, Apodorm, | 15-38 hours | hypnotic | 10 | mg |
| | Pacisyn, Dumolid | | | | |
| Nordazepam | Madar, Stilny | 50-120 hours | anxiolytic | 10 | mg |
| Oxazepam | Serax, Serenid, | 4-15 hours | anxiolytic | 20 | mg |
| | Serepax, Sobril, | | | | |
| | Oxascand, Alopam, | | | | |
| | Oxabenz, Oxapax | | | | |
| Prazepam | Centrax | [36-200 hours] | anxiolytic | 10-20 | mg |
| Quazepam | Doral | 25-100 hours | hypnotic | 20 | mg |
| Temazepam | Restoril, Normison, | 8-22 hours | hypnotic | 15 | mg |
| | Euhypnos | | | | |
| Tetrazepam | Mylostan | 3-26 hours | Skeletal muscle | | |
| | | | relaxant | | |
| Triazolam | Halcion, Rilamir | 2 hours | hypnotic | 0.5 | mg |

Suitably, the pharmaceutical composition is provided in an oral formulation, conveniently, as a pill or capsule comprising clonazepam (1 mg), disulfiram (250 mg), and atropine 0.025 mg contained in a blend, which may be dispensed, for example, in gelatin capsules.

Disulfiram blocks the enzyme aldehyde dehydrogenase and thereby inhibits the oxidation of alcohol at acetaldehyde before it is further broken down to acetic acid⁹. During alcohol metabolism following disulfiram intake, the concentration of acetaldehyde occurring in blood may be 5 to 10 times higher than that found during metabolism of the same amount of alcohol alone. The accumulation of acetaldehyde produces an unpleasant complex of symptoms that immediately blocks further alcohol intake. The disulfiram-ethanol reactions include facial flushing, sweating, headache, nausea, vomiting, tachycardia, palpitations, hyperventilation, hypotension, and dyspnea¹⁰. The disulfiram-ethanol reaction is proportional to the dosage of both chemicals and will persist as long as alcohol remains in the blood plasma. Once having experienced the unpleasant symptoms that result from consuming alcohol in combination with disulfiram, most alcoholics have a strong conditioned aversion to future alcohol intake. People taking disulfiram experience what amounts to enforced sobriety, the essentially total inability to consume alcohol as long as disulfiram levels remain therapeutic.

Disulfiram is absorbed slowly from the gastrointestinal tract. The full effects of disulfiram occur 12 hours after ingestion. Disulfiram is metabolized to diethythiocarbamate and eliminated slowly from the body, and is effective for approximately one to two weeks following administration of the last dose. Prolonged administration of disulfiram does not produce tolerance¹².

Clonazepam is a drug in the family of benzodiazepines. While the exact mechanism of action is unknown, clonazepam is known to enhance the activity of gamma aminobutyric acid (GABA), which is the major inhibitory neurotransmitter in the central nervous system¹³.

Clonazepam is rapidly and completely absorbed after oral administration. The full effects of clonazepam occur within 1 to 4 hours after ingestion. The duration of action is 30 to 40 hours. Clonazepam undergoes hepatic metabolism, and cytochrome P-450 (including CYP3A) may play an important role in its metabolism. It is excreted in the urine as the metabolites clonazepam glucuronide and sulfate conjugates¹³⁻¹⁴.

Atropine sulfate blocks the action of acetylcholine at parasympathetic sites in smooth muscle, secretory glands, and the CNS (although it crosses the blood brain barrier poorly and has CNS effects only at high doses). At effective doses, it increases cardiac output, dries secretions, and antagonizes the effects of histamine and serotonin.

Atropine has a rapid onset of action and its absorption is complete. It is metabolized via the hepatic route with an elimination half-life elimination of 2-3 hours. It is excreted in the urine as 30-50% unchanged drug and the remainder as metabolites¹⁵.

Disulfiram, clonazepam, and atropine are individually FDA approved and have been found to be safe and effective.

The alcohol deterrent properties of disulfiram were first discovered in the United States in 1937 by E. E. Williams¹⁶. Early studies of disulfiram used very high doses (between 1000 and 3000 mg/day) that were associated with significant side effects¹⁷⁻²⁰.

Over the decades, clinical trials have had shortcomings in research design and methodology, and therefore resulted in diverse outcomes²¹⁻²³. The most cited study was a randomized, placebo-controlled clinical trial of disulfiram called the National, Multisite Veterans Administration Study conducted by Fuller et al in 1986²⁴. The three treatment groups were (1) disulfiram (250 mg) with 50 mg of riboflavin; (2) blinded disulfiram (1 mg) with 50 mg of riboflavin; and (3) no disulfiram with 50 mg of riboflavin. The blinded group was an attempt to expose participants to the psychological threat of the disulfiram-alcohol reaction without the actual pharmacological reaction. This study demonstrated that the participants who showed a high degree of treatment adherence were the most successful in maintaining abstinence. However, only 20% of patients who completed the study adhered to disulfiram treatment.

Clonazepam is the drug of choice for treatment of alcohol withdrawal symptoms, and like alcohol is known to increase gabanergic transmission in the central nervous system⁷⁻⁸. When clonazepam is combined with disulfiram, one would expect a significant increase in the percentage of patients who adhere to the treatment because of the reinforcing effects of the benzodiazepine.

The drugs atropine, homatropine (an ester of atropine and mandelic acid), guaifensin, and terpin hydrate all have been successfully used to prevent deliberate misuse of drugs that have an abuse potential, with the first two causing unpleasant anticholinergic side effects and the last two causing nausea and vomiting. For example, the anti-diarrheal Lomotil is a combination of the opiate diphenoxylate with subclinical atropine, with a resultant low abuse liability.

The AUDIT screening tool will be used in the initial assessment and screening for alcohol use disorders and at all follow-up visits. AUDIT has been validated as an effective screening tool for alcohol use disorders²⁵.

The initial studies submitted to the FDA for approval of the drug used doses as chosen in this study. The current FDA recommended average daily dose of disulfiram is 250 mg, with a range of 125 to 500 mg (maximum daily dosage). In addition, past studies on disulfiram at that dose prove that this dosing range is safe and well-tolerated²⁶⁻²⁷.

Clonazepam's indications are for treatment of seizures and panic disorder, but it is widely used for anxiety and insomnia, like many other clinically available benzodiazepines. It is widely believed by many medical experts to have less abuse potential than other benzodiazepines, due to its pharmacokinetics (i.e. long elimination half-life of 30-40 hours). The target dose of clonazepam for treatment of seizures is individualized depending on patient response, with a maximum of 20 mg/day. The target dosing of clonazepam for treatment of panic disorder is 1 mg/day, with a range of 0.25 mg/day to a maximum of 4 mg/day¹³. A multicenter, parallel-group, placebo-controlled, fixed-dose study investigated the efficacy, safety and dosing characteristics for clonazepam in patients with panic disorder. Four hundred thirteen patients were randomly assigned to receive placebo or one of five fixed daily doses of clonazepam (0.5 mg, 1.0 mg, 2.0 mg, 3.0 mg, and 4.0 mg). Daily doses of 1.0 to 2.0 mg were found offer the best balance of therapeutic benefit and tolerability²⁸.

The dose of atropine in the pharmaceutical composition of the present invention is based on clinical studies done for combination medicines (such as Lomotil and Motofen) that use the same dose of atropine as a deterrent for abuse³⁰. Studies using this same dose of atropine sulfate (0.025 mg) were submitted to the FDA, prior to approval of Lomotil and Motofen, and atropine sulfate was found to be safe and effective. It is expected that atropine sulfate could be administered as an abuse aversive agent at a subclinical dose ranging from about 0.025 mg to about 0.05 mg per unit dose of medicine (i.e. per pill).

The risk of addiction to clonazepam is inherent in this proposed combination agent, and dependence on clonazepam is possible. Drug substitution underlies the basis for treatment of numerous other addictions, including methadone and buprenorphine to treat heroin addiction and nicotine patches to treat tobacco addiction. It is accepted that the medical and psychosocial problems and risks associated with alcohol abuse far outweigh the risk of benzodiazepine dependence. In addition, clonazepam can only be obtained by prescription or illicit drug purchase, whereas no barrier exists for alcohol acquisition, making it harder for the addict to acquire clonazepam as opposed to his/her drug of choice (alcohol).

Hepatotoxicity has been associated with high doses of disulfiram, but not at the dose used in this study (250 mg/day to a maximum of 500 mg/day). Patients with acute, severe or end-stage liver disease will be excluded from the study.

The efficacy of a specific pharmaceutical composition according to the present invention comprising clonazepam 1 mg, disulfiram 250 mg, atropine 0.025 mg (“the combination pill”) in treating alcohol addiction will be evaluated in a phase II, open label, non-randomized study including 20 subjects. The expected duration of subject participation is 6 months.

Patients will be recruited and screened from the primary investigator's clinical practice and by referral from other clinicians, particularly the primary care group and the inpatient academic hospitalists, who see a particularly high burden of alcoholism. At the initial consultation, patients who qualify for the study will be consented, asked to fill out the AUDIT questionnaire, and given a urine toxicology screen to rule out polysubstance abuse. Each subject will receive seven capsules of active drug, will take the first dose under medical supervision, and will be asked to participate in abstinence psychosocial support of his choosing (AA, or other outpatient treatment program). The subjects will be educated about the disulfiram reaction and will be warned against the consumption of any alcohol. An outpatient follow-up appointment in 1 week or less will be made with the primary investigator (or a covering internal medicine colleague who will be familiar with the study protocol if the PI is on vacation). Every necessary action will be undertaken to insure timely follow-up including automatic overbooking of study patients if slots are not available, so patients do no have any lapse in the study drug.

At the first follow-up, patients undergo a brief repeat history and physical exam, and a repeat urine toxicology screen. If it is positive for anything other than benzodiazepines, the patient will be withdrawn from the study. If positive for only benzodiazepines, the patient will undergo a repeat AUDIT questionnaire and an additional study questionnaire focusing on desire to drink, worrisome side effects, psychosocial functioning, and participation in psychosocial treatment. If the combination pill is not fully suppressing withdrawal, the patient will be instructed to increase his dose by taking two pills by mouth once daily (maximum daily dosage of combination pill). Patients will be given a prescription for a 30 day supply of the combination drug to be filled at the compounding pharmacy. They will be encouraged to continue psychosocial support on every visit.

Follow-up appointments will thereafter be monthly for a total of 6 months. Each follow-up will include history and physical assessment by the investigator, the AUDIT questionnaire, the additional study questionnaire, and a 1 month prescription for the combination drug. At any point if the combination pill is found to be ineffective, the patient will be instructed to increase their dose by taking two pills by mouth once daily.

Patients withdrawn from the study (by personal or study team decision) will be asked to stop taking the combination pill. They will undergo a taper of clonazepam (not combined with other medications). The clonazepam dose will be tapered gradually, with a decrease of 0.125 mg bid every week as the patient tolerates, until the drug is completely withdrawn.

At the end of the study, patients who chose to continue the combination pill may do so, and will continue to be followed the primary investigator. Patients who chose to discontinue the combination pill will undergo a clonazepam taper as described above.

Primary endpoint to be analyzed in the study is the efficacy of the combination pill to prevent subjects from consuming alcohol (relapse).

The AUDIT screening tool will be used in the initial assessment and screening for alcohol use disorders and at all follow-up visits. AUDIT has been validated as an effective screening tool for alcohol use disorders²⁵.

Secondary endpoints to be analyzed in the study are to safety and tolerability of three agents (disulfiram, clonazepam, and atropine) together in the combination drug.

Subjects must meet the following criteria to be eligible for study enrollment if they:
1. Are male or, for females, are nonlactating, nonpregnant, and if of childbearing potential, using an acceptable method of birth control.
2. Are between 18 and 50 years of age.
3. Suffer from alcohol abuse or dependence and desire to stop drinking.
4. Are willing and able to come to all study clinic appointments.
5. Are willing and able to give informed consent, fill out all questionnaires pertaining to study and provide specimen (i.e., urine) sample.

Criteria that would exclude a subject from study enrollment include:
1. Any prior history of hypersensitivity to disulfiram, clonazepam or atropine.
2. Patients with liver disease (acute, severe or end-stage), kidney disease (acute or chronic), myocardial disease and previous coronary occlusion, narrow-angle glaucoma, psychosis, seizure disorders, or pregnancy.
3. Patients concomitantly taking trimethoprim-sulfamethoxazole, isoniazid, MAO inhibitors, metronidazole, TCAs, phenytoin, and theophylline.
4. A severe physical dependence on alcohol defined by recent intake of greater than 6 alcoholic drinks daily until medical detoxification has taken place.

Subjects will be recruited for the study from the investigator's clinical practice and referring clinicians via advertisement.

Subjects may be withdrawn from the study prior to the expected completion for safety reasons, failure of subject to adhere to protocol requirements, subject consent withdrawal. If a patient is withdrawn, they will be instructed to stop taking the investigational study pill. The patient will be placed on alternate therapy of clonazepam (not combined with any other drug) to be tapered with a decrease of 0.125 mg bid every week as tolerated, until the drug is completely withdrawn.

Survival data on all subjects lost to follow-up will be collected. Patients lost to follow-up will have no fewer than two phone calls to subject, a phone call to next of kin and a certified letter sent to patient.

The combination pill comprises clonazepam 1 mg, disulfiram 250 mg, and atropine 0.025 mg blended and placed in a gelatin capsule, and is taken by mouth daily for the duration of the study. Patients for whom this dose is not sufficient as evidenced by continued withdrawal symptoms may increase dose to two pills by mouth once daily (the maximum daily dose).

Subject compliance with the treatment regimen will be assessed and tracked by the primary investigator at all study visits. Any subject who is significantly non-compliant with the study treatment regimen will be withdrawn.

Concomitant medicines and therapies permitted during the study include anything the patient was receiving prior to initiating the study. Patients on long-term warfarin will require close monitoring of their INR and possible adjustment of their warfarin dosage.

Close monitoring is necessary for any patient previously on CNS depressants, psychoactive agents, or drugs with anti-cholinergic activity.

Concomitant medicines not permitted during the study include trimethoprim-sulfamethoxazole, isoniazid, MAO inhibitors, metronidazole, TCAs, phenytoin, and theophylline.

Gelatin capsules containing study drug, seven per bottle will be dispensed to patient.

The bottle will come with the following warning:
DO NOT TO EXCEED THE RECOMMENDED DOSAGE AND TO KEEP STUDY MEDICINE OUT OF THE REACH OF CHILDREN AND INA CHILD-RESISTANT CONTAINER. THE CONSEQUENCES OF OVERDOSAGE MAY INCLUDE SEVERE RESPIRATORY DEPRESSION AND COMA, POSSIBLY LEADING TO PERMANENT BRAIN DAMAGE OR DEATH CONSUMPTION OF ALCOHOL IN ANY FORM WITH THIS MEDICATION WILL PRODUCE VIOLENT SICKNESS, WHICH IN EXTREME CASES MAY BE FATAL.
Clonazepam and atropine sulfate may produce drowsiness or dizziness. Use caution regarding activities requiring mental alertness, such as driving or operating dangerous machinery. Potentiation of the action of alcohol, barbiturates and tranquilizers with concomitant use of clonazepam and atropine sulfate may occur.

The physician should also provide the patient with other information, as appropriate, provided by the pharmacy regarding all three active drugs contained within the study medication.

This study is not blinded.

University of Chicago inpatient pharmacy will compound the study drug from readily available FDA approved single agent tablets.

Study drug will be stored in a tight, light-resistant container at controlled room temperature 15°-30° C. (59°-86° F.). The study drug will be kept in the University of Chicago Pharmacy.

Patients will be recruited by the investigator's outpatient clinical practice, referring clinicians from the Medicine Department aware of the study, or through clinicians made aware of the study via advertisement.

### Visit 1—The Initial Consultation

In the outpatient setting at the initial consultation individuals who qualify for the study and agree to participate will need to sign the consent form and fill out the AUDIT questionnaire (see attachment D). They will be given a seven day supply of the combination pill at the time of the initial consultation and encouraged to participate in psychosocial treatment of their choice (AA, outpatient treatment, etc). They also will be told to call the scheduling center for an appointment to be seen exactly one week from the initial consultation and inform the scheduler the appointment is a study follow-up. The scheduling center will have approval from the primary investigator to overbook as necessary so that the first study follow-up is one week from the initial consultation.

Inpatient candidates referred to the study will be seen as an inpatient consult by the investigator's clinical study nurse the day of or day prior to their discharge. In the inpatient setting at the initial consultation, the clinical nurse will identify if the patient qualifies and agrees to participate. The patient will need to sign the consent form and fill out the AUDIT questionnaire at the time of the initial inpatient consultation. They will be given a seven day supply of the combination pill at the time of the initial consultation and encourage to participate in psychosocial treatment of their choice (AA, outpatient treatment, etc). They also will be told to call the scheduling center for an appointment to be seen exactly one week from the initial consultation and inform the scheduler the appointment is a study follow-up. The scheduling center will have approval from the primary investigator to overbook as necessary so that the first study follow-up is one week from the initial consultation. Patient will be cautioned against drinking alcohol or consuming any illicit substances during the clinical trial.

### Visit 2—First Follow-Up

At the first follow-up, patients will undergo a history and physical exam by the primary investigator, and a urine toxicology screen. If it positive for anything other than the study benzodiazepine, the patient will be withdrawn. If negative for anything other than benzodiazepines, the patient will fill out an AUDIT questionnaire and an additional study questionnaire focusing on desire to drink, worrisome side effects, psychosocial functioning, and participation in psychosocial treatment. If the combination pill is found to be ineffective, the patient will be instructed to increase their dose by taking two pills by mouth once daily (maximum daily dosage of combined agent). Patients will be given a prescription for a 30 day supply of the combination drug to be filled at the compounding pharmacy. They will be told to call the scheduling center for a follow-up appointment to be seen one month from current visit. Finally, they will be encouraged to continue psychosocial treatment.

### Visit 3—Month #1

At the second follow-up, patient will undergo a thorough assessment and history and physical exam by the primary investigator. The patient will fill out an AUDIT questionnaire and the additional study questionnaire. If the combination pill is found to be ineffective in suppressing withdrawal symptoms, the patient will be instructed to increase their dose by taking two pills by mouth once daily (maximum daily dosage of combined agent). Patients will be given a prescription for a 30 day supply of the combination drug to be filled at the University of Chicago pharmacy. They will be told to call the scheduling center for a follow-up appointment to be seen 1 month from current visit. Finally, they will be encouraged to continue psychosocial treatment.

### Visit 4—Month #2

Same as above.

### Visit 5—Month #3

Same as above.

### Visit 6—Month #4

Same as above.

### Visit 7—Month #5

Same as above.

### Visit 8—Last Study Visit

Same as above. Patient will be informed that the study will conclude in two to three weeks. Patients will have option to continue taking the combination pill or to discontinue it. If they chose stopping the combination pill, they will undergo a clonazepam taper (not combined with other study medications). The clonazepam dose will be discontinued gradually, with a decrease of 0.125 mg bid every week, as tolerated, until the drug is completely withdrawn.

### Statistical Plan

### Sample Size Determination

This is a limited, small pilot study to establish proof of concept of this medication.

### Statistical Methods

This is a limited, small pilot study to establish proof of concept of this medication and to assess doses and effects.

### Subject Population(s) for Analysis

Protocol-compliant population.

Each of the following publications is incorporated by reference in its entirety:
1. Bridget F. Grant, Ph.D., Ph.D., Deborah A. Dawson, Ph.D., Frederick S. Stinson, Ph.D., S. Patricia Chou, Ph.D., Mary C. Dufour, M.D., M. P. H. and Roger P. Pickering, M. S., 2006. The 12-Month Prevalence and Trends in DSM-IV Alcohol Abuse and Dependence: United States, 1991-1992 and 2001-2002. National Institute on Alcohol Abuse and Alcoholism, Bethesda, Md.
2. Weinrieb R M, O'Brien C P. Diagnosis and treatment of alcoholism. In: Dunner D L, eds. Current Psychiatric Therapy. 2nd ed. Philadelphia, Pa.: Saunders; 1997:130-139.
3. Fuller R K, Branchey L, Brightwell D R, et al. Disulfiram treatment of alcoholism. A Veterans Administration Cooperative Study. JAMA. 1986; 256:1449-1455.
4. Landabaso M A, Iraurgi I, Sanz J, et al. Naltrexone in the treatment of alcoholism. Two-year follow up results. Eur J Psychiatry. 1999; 13(2):97-105.
5. Petrakis I L, Poling J, Levinson C, et al. VA New England VISN I MIRECC Study Group. Naltrexone and disulfiram in patients with alcohol dependence and comorbid psychiatric disorders. Biol Psychiatry. 2005; 57:1128-1137.
6. Besson J, Aeby F, Kasas A, et al. Combined efficacy of acamprosate and disulfiram in the treatment of alcoholism: a controlled study. Alcohol Clin Exp Res. 1998; 22: 573-579.
7. Lejoyeux M, Solomon J, Ades J. Benzodiazepine treatment for alcohol-dependent patients. Alcohol Alcohol. 1998 November-December; 33(6):563-75. Review.
8. Williams D, McBride A J. The drug treatment of alcohol withdrawal symptoms: a systematic review. Alcohol Alcohol. 1998; 33(2):103-15. Review.
9. Johansson B. A review of the pharmacokinetics and pharmacodynamics of disulfiram and its metabolites. Acta Psychiatr Scand. 1992; 86: 15-26.
10. Ayerst Laboratories. Antabuse (Disulfiram) in Alcoholism. Philadelphia, Pa.: Wyeth-Ayerst Company; 2001.
11. Disulfiram. In: AltCareDex® System [Internet database]. Greenwood Village, Colo.: Thomson Micromedex. Updated periodically.
12. Disulfiram. In: UpToDate, Waltham, MA, 2006.
13. Clonazepam. In: AltCareDex® System [Internet database]. Greenwood Village, Colo.: Thomson Micromedex. Updated periodically.
14. Clonazepam. In: UpToDate, Waltham, MA, 2006.
15. Clinical data from studies performed by GD Searle LLC on Lomotil and Amarin Pharmaceuticals on Motofen prior to FDA approval.
16. Williams E E. Effects of alcohol on workers with carbon disulfide. JAMA. 1937; 109:1472-1473.
17. Fuller R K, Gordis E. Does disulfiram have a role in alcoholism treatment today? Addiction. 2004; 99:21-24.
18. Martensen-Larsen O. Psychotic phenomena provoked by tetraethylthiuram disulfide. Q J Stud Alcohol. 1951; 12:206-216.
19. Guild J, Epstein N B. Psychosis during the treatment of alcoholism with tetraethylthiuram disulfide. Q J Stud Alcohol. 1951; 12:360-365.
20. Jacobsen E. Deaths of alcoholic patients treated with disulfiram (tetraethylthiuram disulfide) in Denmark. Q J Stud Alcohol. 1952; 13:16-26.
21. Lundwall L, Baekeland F. Disulfiram treatment of alcoholism. J Nerv Ment Dis. 1971; 153(6):381-394.
22. Garbutt J C, West S L, Carey T S, et al. Pharmacological treatment of alcohol dependence. A review of the evidence. JAMA. 1999; 281:1318-1325.
23. Mottin J L. Drug-induced attenuation of alcohol consumption: a review and evaluation of claimed, potential or current therapies. Q J Stud Alcohol. 1973; 34:444-472.
24. Fuller R K, Branchey L, Brightwell D B, et al. Disulfiram treatment of alcoholism. A Veterans Administration Cooperative Study. JAMA. 1986; 256:1449-1455.
25. Coulton S, Drummond C, James D, et. al. Opportunistic screening for alcohol use disorders in primary care: comparative study. BMJ. 2006; 332(7540):511-7.
26. Banys P. The clinical use of disulfiram (Antabuse): a review. J Psychoactive Drugs. 1988; 20:243-260.
27. Kristenson H. How to get the best out of Antabuse. Alcohol Alcohol. 1995; 30:775-783.
28. Rosenbaum J F, Moroz G, Bowden C L. Clonazepam in the treatment of panic disorder with or without agoraphobia: a dose-response study of efficacy, safety, and discontinuance. J Clin Psychopharmacol. 1997; 17(5): 390-400.
29. Anton R F, O'Malley S S, et al. Combined pharmacotherapies and behavioral interventions for alcohol dependence: the COMBINE study: a randomized controlled trial. JAMA. 2006 May 3; 295(17):2003-17.
30. Kasich, A M. Treatment of diarrhea in irritable colon, including preliminary observations with a new antidiarrheal agent, diphenoxylate hydrochloride (Lomotil). Am J Gastroenterol. 1961 January; 35:46-9.
31. Carroll, K M, Fenton, L. R. et al. Efficacy of Disulfiram and Cognitive Behavior Therapy in Cocaine Dependent Outpatients. Arch Gen Psychiatry. 2004 March; 61:264-272.
32. Carroll, K M, Nich, C, et al. One-year follow-up of disulfiram and psychotherapy for cocaine-alcohol users: sustained effects of treatment. Addiction. 2000; 95(9):1335-1349.
33. Carroll, K M, Nich, C, et al. Treatment of cocaine and alcohol dependence with psychotherapy and disulfiram. Addiction. 1998; 93(5):713-728.
34. National Survey on Drug Use and Health, 2004 and 2005, SAMHSA, Office of Applied Sciences.

It is claimed:

1. A pharmaceutical composition comprising a benzodiazepine, an alcohol aversive agent, and an abuse aversive agent.

2. The composition of claim 1, wherein the alcohol aversive agent is disulfiram, citrated calcium carbamide, or coprine.

3. The composition of claim 1, wherein the alcohol aversive agent is disulfiram.

4. The composition of claim 1, wherein the benzodiazepine is selected from the group consisting of: alprazolam, bromazepam, chlordiazepoxide, clobazam, clonazepam, clorazepate, diazepam, estazolam, flunitrazepam, flurazepam, halazepam, ketazolam, loprazolam, lorazepam, lormetazepam, medazepam, midazolam, nitrazepam, nordazepam, oxazepam, prazepam, quazepam, temazepam, and triazolam.

5. The composition of claim 1, wherein the benzodiazepine is clonazepam.

6. The composition of claim 1, wherein the abuse aversive agent is atropine.

7. The composition of claim 1, wherein the composition comprises disulfiram in an amount effective to cause alcohol aversion in a subject ingesting alcohol and clonazepam in an amount effective to reduce at least one symptom associated with alcohol withdrawal.

8. The composition of claim 1 wherein the abuse aversive agent is in a subclinical dose in unit dose form, and in a dose effective to cause aversive symptoms when the composition is taken in excess of the prescribed dosage.

9. The composition of claim 8, wherein the abuse aversive agent is atropine.

10. A method of preventing alcohol consumption and reducing at least one symptom of alcohol withdrawal in a subject comprising administering to the subject the composition of claim 1 in an amount effective to reduce at least one symptom of alcohol withdrawal and to cause alcohol aversion.

11. The method of claim 10, wherein the composition is administered in an amount effect to reduce at least one symptom of alcohol withdrawal and to cause alcohol aversion when the subject ingests alcohol.

12. The method of claim 10, wherein the pharmaceutical composition comprises disulfiram, clonazepam, and atropine.

13. A method of preventing cocaine consumption in a subject comprising administering to the subject a pharmaceutical composition comprising a benzodiazepine and an alcohol aversive agent in an amount effective to reduce cocaine use.

14. The method of claim 13, further comprising a subclinical dose of an abuse aversive agent.

15. The method of claim 13, wherein the alcohol aversive agent is disulfiram, citrated calcium carbamide, or coprine.

16. The method of claim 15, wherein the alcohol aversive agent is disulfiram.

17. The method of claim 13, wherein the benzodiazepine is clonazepam.

18. The method of claim 14, wherein the abuse aversive agent is atropine.

19. The method of claim 13, wherein the pharmaceutical composition comprises disulfiram and clonazepam.

20. A method of preventing cocaine and alcohol consumption by a subject comprising administering to the subject a pharmaceutical composition comprising a benzodiazepine and an alcohol aversive agent in an amount effective to cause alcohol aversion and reduce at least one symptom of withdrawal.

21. A method of preventing at least one of consumption of an addictive substance by a subject and symptoms of withdrawal from an addictive substance in a subject comprising administering to the subject a pharmaceutical composition of claim 1 in an effective amount.
